(19) United States
(12) Patent Application Publication
(10) Pub. No.: US 2016/0287484 A1
(43) Pub. Date: Oct. 6, 2016

(54) Dry Shampoo Composition for Hair

(71) Applicant: Emma Neame, London (GB)

(72) Inventor: Emma Neame, London (GB)

(21) Appl. No.: 15/036,708

(22) PCT Filed: Nov. 17, 2014
(86) PCT No.: PCT/GB2014/000473

§ 371 Date: May 13, 2016

(30) Foreign Application Priority Data
Nov. 15, 2013 (GB) 1320303.9

(51) Int. Cl. A61K 8/02; A61K 8/19; A61Q 5/12; A45D 33/00; A61Q 13/00; A61K 8/97; A61K 8/31; A45D 33/02; A61Q 5/02; A61K 8/73

(52) U.S. Cl. CPC A61K 8/022; A61Q 5/02; A61K 8/19; A61Q 5/12; A61K 8/732; A61Q 13/00; A61K 8/97; A61K 8/31; A45D 33/02; A45D 33/005; A61K 2800/87; A61K 2800/30

(57) ABSTRACT

The invention provides a dry shampoo composition for cleaning hair comprising: 4 to 35% baking soda and 65 to 96% starch. The constituent proportions are expressed in terms of proportions by weight relative to the total weight of the composition. Optionally the composition may also contain a conditioning agent and/or a fragrance.

### FIELD OF THE INVENTION

The present invention relates to a dry shampoo composition primarily intended for quickly cleansing a child's hair when wet washing is neither desirable nor appropriate. Although the composition is primarily intended as a hair cleaning composition, its use is not exclusively so limited.

The present composition is particularly suitable for use on sensitive and/or young skin or those persons with a sensitive scalp. The present invention can be used as a dry shampoo for the rapid cleansing of soiled hair when there is not time enough to wet wash and dry the hair or when one does not want to wet wash the hair too often.

### BACKGROUND

For many years, dry hair cleaning compositions have been available for the rapid cleansing of soiled hair. These known compositions can be used on dry hair in between wet washing, for either freshening up the hair or to remove excess oils and other undesirables found on soiled hair. These known compositions are particularly useful when time is short and one cannot wash the hair using wet shampoo or other hair and skin cleaning compositions and then dry the hair, particularly if it is very long and the hair requires half an hour or so to carry out the procedure. Although these known compositions contain chemicals especially designed to cleanse the hair effectively without the use of water, the chemicals used may be harsh and may not be suitable for use on sensitive or young skin or scalp.

Many parents will have experienced their young child choosing exactly the wrong moments to soil its hair, most often by the act of putting their dirty hands on their head when eating or when being fed. This behaviour may start when a child is weaned onto solid foods, at about 6 months old or so. There is usually no time to bath the child to wash out whatever they have put into their hair. Whilst a quick wipe with a wet flannel will suffice to clean their dirty hands and face, this method is hardly suitable or desirable for cleaning their hair, because the flannel will just smear the foodstuff and other undesirables into the hair rather than removing it completely.

It has been suggested to use talcum powder to remove grease and foodstuffs from a child's hair, but this is considered highly dangerous as a child can breathe the talcum powder into its lungs.

Furthermore, it is advised that children's hair not be wet washed too often, as wet washing removes natural conditioning agents found on the scalp and skin, leading to skin and hair which is over dry.

The composition of the present invention can be used in between wet washes to keep the hair clean and soft and is particularly useful when a child has become sweaty or dirty through play, or as mentioned above, has put foodstuffs into its hair when eating or when being fed.

The present invention aims to provide an alternative composition to the often harsh chemical laden compositions currently on the market. The composition of the present invention aims to provide an effective combination of ingredients so that the composition can be safely used on young and/or sensitive skin, the composition is effective in ease of application, cleaning of the hair, ease of removal without leaving a residue on the hair and leaves the hair feeling soft after use.

The present invention is presented as a dry composition which may be applied onto the hair, for example by directly sprinkling onto the hair, or which may be sprinkled onto the hands and then applied onto the hair. The applied composition is then distributed through the hair, for example by massaging into the hair or ruffling the hair, to remove grease, oil and other undesirables, then removed from the hair using, for example, a comb or brush.

The present composition can also be used by those who have hair which becomes greasy very quickly and wet washing is not an option. This may apply particularly to those undergoing puberty, one of the symptoms of which is an over production of sebum on hair and skin which can render the hair and skin very greasy only a few hours after wet washing.

### SUMMARY OF THE INVENTION

The composition of the present invention comprises mainly dry ingredients and comprises at least baking soda and starch. The composition may also comprise a conditioning agent to soften the hair.

The composition may also include a fragrance, which may be provided in a dry powder form or as an essential oil. Where an essential oil is used, the composition is still in a dry powder form since the fragrance comprises a very small percentage in proportion by weight relative to the total weight of the composition, and is evenly distributed through the bulk volume of the other constituents without rendering the dry powder composition oily.

In a first aspect of the invention, there is provided a dry hair shampoo composition. The composition comprises 4 to 35% baking soda, the remainder or balance being starch, incidental constituents and impurities. The constituent proportions are expressed in terms of proportions by weight relative to the total weight of the composition.

The term “dry hair shampoo” as used herein, is to be interpreted as meaning dry shampoo used for cleaning hair of any condition, rather than meaning a shampoo used for cleaning hair which is perceived to be of a “dry” condition.

In a second aspect of the invention there is provided a dry hair shampoo composition comprising: 1 to 20% baking soda, 3 to 15% conditioning agent; the remainder or balance being starch, incidental constituents and impurities. The constituent proportions are expressed in terms of proportions by weight relative to the total weight of the composition.

In a third aspect of the invention there is provided a dry hair shampoo composition comprising: starch; a conditioning agent and baking soda, wherein: the starch component is present in proportions by weight relative to the total weight of the composition in the range 72 to 96%: and the ratio of conditioning agent to baking soda in proportions by weight relative to the total weight of the composition is in the range 1.5:1 to 1:2.5. The ratio is the ratio of conditioning agent to baking soda.

In another aspect of the invention there is provided a dry hair shampoo composition, the composition comprising the following constituents in proportions by weight relative to the total weight of the composition: 4 to 35% baking soda; 64 to 95.99% starch and 0.01 to 5% fragrance.

A further aspect of the invention provides a dry hair shampoo composition comprising the following constituents in proportions by weight relative to the total weight of the composition: 1 to 20% baking soda; 3 to 15% conditioning agent; 0.01 to 5% fragrance; and the remainder or balance being starch, incidental constituents and impurities.

Yet a further aspect of the invention provides a dry hair shampoo composition comprising: starch; a conditioning agent and baking soda; and fragrance, wherein: the starch component is present in proportions by weight relative to the total weight of the composition in the range 67 to 95.99%: the fragrance is present in proportions by weight relative to the total weight of the composition in the range 0.01 to 5%; and the ratio of conditioning agent to baking soda in proportions by weight relative to the total weight of the composition is in the range 1.5:1 to 1:2.5. The ratio is the ratio of conditioning agent to baking soda.

The fragrance is optionally included to impart a fresh scent to the hair and comprises an essential oil or a dry powder extract of one or more of the following: chamomile; lavender; yarrow; dill; coriander; neroli; mandarin; grapefruit; aniseed; beetroot; calendula; neeme; lemon; ginger; rosemary; and cocoa.

Where an essential oil is used, it must be carefully selected since use of essential oils on very young children is ill advised. This is because many essential oils contain alcohols which may over-dry the skin and other essential oils contain irritants which may be harmful to sensitive and/or young skin.

Essential oils should not be used on children under around 6 months since many essential oils can irritate the skin.

For example, essential oils suitable for use on children aged from around 6 to 12 months may be selected from the group comprising: chamomile; lavender; yarrow; dill; coriander; neroli; mandarin; grapefruit; aniseed; beetroot; and calendula.

For children older than 2 years, lemon, ginger and rosemary essential oils may be used.

Other fragrances suitable for use in the composition of the present invention include, but are not limited to, cocoa and neeme oil.

Neeme oil has been shown to assist in ridding hair of head lice and would be suitable for children of school age, where such head lice infestations can be prevalent.

Of the above fragrances, chamomile is the mildest and most suitable for all ages of children above 6 months and persons with sensitive scalps or skin. Some of these fragrances also lend themselves as conditioning agents in the composition. For example, chamomile may serve a dual purpose of softening the hair as well as providing a pleasant odour to the composition and thus to the hair.

Some of these fragrances can also impart a colour to the composition. For example, cocoa and beetroot imparts a pleasant colour to the composition as well as imparting a pleasant scent.

The starch, in the composition, acts as an absorbing agent to remove excess moisture and/or grease. The starches tested were corn starch, potato starch, rice flour, oat flour and tapioca flour. Out of the potato, rice and corn starches, it was found that corn starch absorbs the most grease, oils and/or moisture and is the most preferable for use in the composition as it also has little or no odour. In the case of oat and tapioca flours, these serve to act as flowing agents or anti-caking agents, as well as absorbing excess grease and moisture, which assist with the application and removal of the dry composition by imparting a fine consistency to the composition.

It was found that that a combination of corn starch with tapioca flour were the best starches for imparting a fine consistency to the powder and thus making the composition easy to sprinkle onto the hair and also easy to remove. The tapioca flour also assists in keeping the composition dry and in a usable form for many months.

The starch is present in the composition in a dry powder form.

Preferably the total starch comprises a mixture of 95 to 99.99% tapioca flour and 0.1 to 5% corn starch in proportions by weight relative to the total weight of the starch. This range is useful in terms of how easily it can be applied to the hair and leaves little or no residue on the hair when removed after cleaning the hair.

Sodium bicarbonate, or as commonly known, baking soda or Bicarbonate of soda, acts as the cleaning agent in the composition and is preferably present in a dry powder form. The baking soda serves to cleanse the hair effectively and also acts to absorb odours.

The conditioning agent is preferably in the form of a dry powder. The conditioning agent acts to soften the hair. Preferably, the conditioning agent used in the composition of the present invention is cucumber extract but coconut oil powder, or a combination of both may also be used. Other conditioning agents may also be used.

Preferably the composition comprises: starch in the range 84 to 96%; the baking soda in the range 2 to 12% and the conditioning agent in the range from 2 to 4%, all in proportions by weight relative to the total weight of the composition.

Preferably the composition comprises starch being present in the range 77 to 84%; the baking soda being present in the range 10 to 15% and the conditioning agent being present in the range from 6 to 8%, all in proportions by weight relative to the total weight of the composition.

Preferably in the composition the starch comprises 0.1 to 5% cornstarch and 95 to 99.9 tapioca flour and the total starch is present in the range 86 to 77%; the baking soda is present in the range 8 to 15% and the conditioning agent is present in the range from 6 to 8%, all in proportions by weight relative to the total weight of the composition.

Preferably in the composition, the starch is present in the range 83 to 68%; the baking soda is present in the range 5 to 15% and the conditioning agent is present in the range from 12 to 17%, all in proportions by weight relative to the total weight of the composition.

A further aspect of the invention provides a container containing the composition described above, the container being provided with means for applying the composition hair, wherein the means for applying comprises holes in the top of the container, through which the composition is dispensed.

Another aspect of the invention provides a sachet containing the composition described above. The sachet preferably contains about 0.2 to 3 g of the composition.

Yet another aspect of the invention provides a method of cleaning hair, using the composition described above, the method including the steps of: applying the dry composition onto the hair, for example, by sprinkling the composition onto the hair; and removing the composition from the hair.

The composition can be applied by sprinkling the composition, by dispensing pinches of the composition or dispensing into the palm of a hand and then applying to the hair.

The composition can be distributed though the hair, for example by massaging the hair.

The composition can be removed from the hair by brushing or combing, for example.

The composition is preferably used on children aged around 6 months to 15 years.

Further aspects of the invention are set out in the appended claims.

### BRIEF DESCRIPTION OF THE DRAWINGS

FIG. 1 depicts a child who, during feeding, has deposited food into its hair by touching its head with dirty hands;

FIG. 2 illustrates the use of the composition of the present invention wherein the composition is dispensed from a lidded container;

FIG. 3 illustrates the use of the composition of the present invention, wherein the composition is dispensed from a sachet; and

FIGS. 4 to 8 are graphs of the results of tests of samples of the composition in which scores out of 10 have been assigned for effectiveness in cleaning and hair feel after treatment for different percentages of starch and baking soda present, relative to the total weight of the composition.

### DETAILED DESCRIPTION

### Method of Use

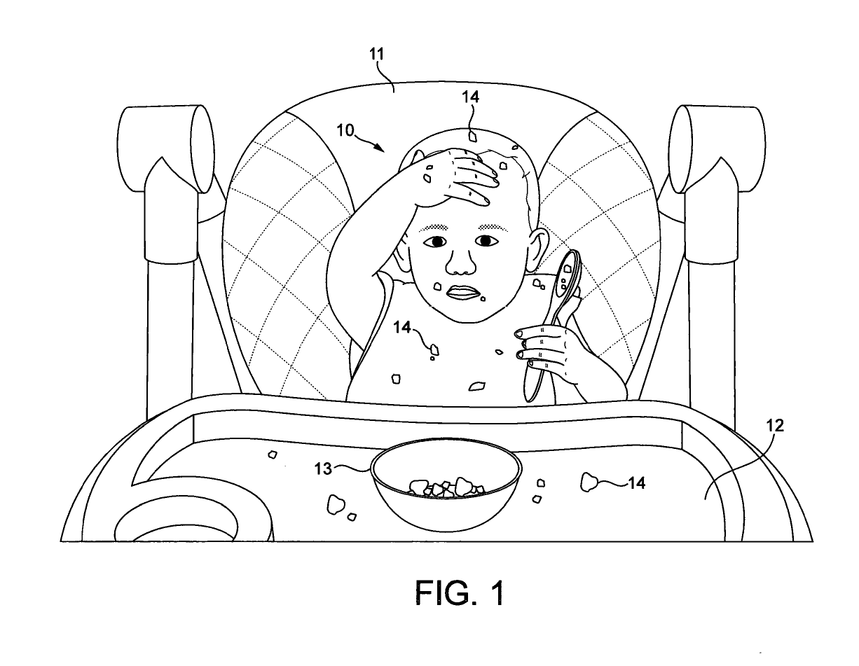

FIG. 1 shows a child 10 who is sitting in a high chair 11, equipped with a table 12. A bowl 13 of food 14 is placed in front of the child 10. The child 10, during feeding, has deposited food 14 onto the surface of the table 12, its clothes and into its hair by touching its head with dirty hands.

After the child has finished feeding, its hands and the table 12 can be wiped clean with a flannel. The child's clothes can also be changed; however the food in its hair would smear if a flannel were used to clean it.

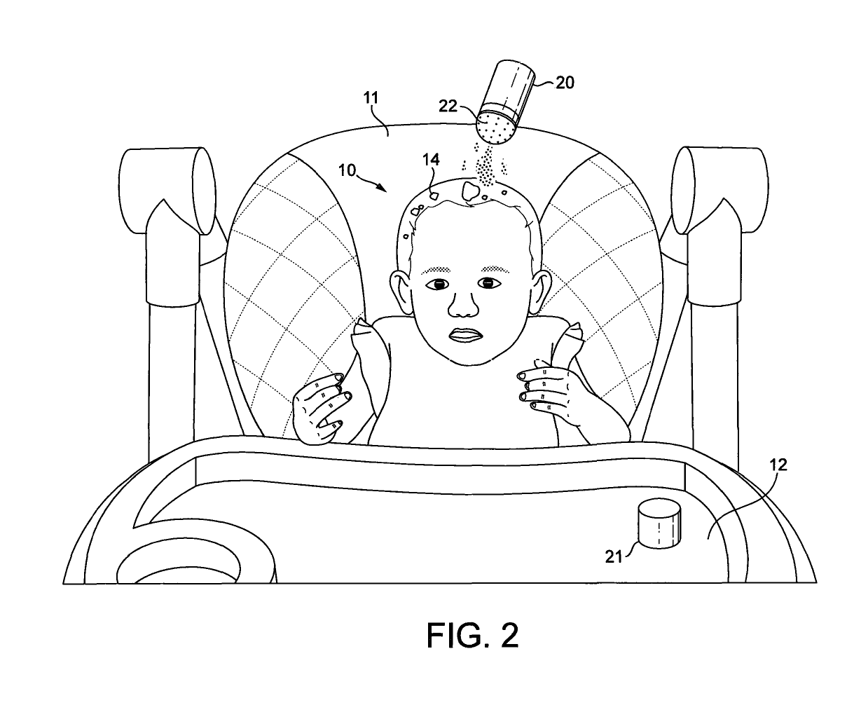

FIG. 2 illustrates one method of use of the composition of the present invention. The composition is stored in a container 20 with a perforated top which, for the purposes of storage, is provided with a lid 21. In the illustrated example, the container has perforations 22 in the top, through which the powder can be sprinkled onto the head. Alternatively, the powder could have been sprinkled onto the hand and then applied to the hair of the child. The container was given 3 shakes to sprinkle the composition onto the hair. Of course, for children with longer hair, or more dirt, more shakes of the container would be required. The container typically contains 20 g of the composition which is suitable for about 50 or so uses of the container, since only a very small quantity of the composition is required to be dispensed from the container for a single cleansing operation.

A typical quantity of the composition required for cleaning a child's hair is around 0.2 to 3 g. Alternatively the range may be 0.2 to 2.5 g. Alternatively the range may be 0.2 to 2.0 g. Alternatively the range may be 0.2 to 2 g. Alternatively the range may be 0.2 to 1.5 g. Alternatively the range may be 0.2 to 1 g. Alternatively the range may be 0.2 to 0.5 g. Another range may be 0.4 to 3 g. Alternatively the range may be 0.6 to 3 g. Alternatively the range may be 0.8 to 3 g. Alternatively the range may be 1.0 to 3 g. Alternatively the range may be 1.2 to 3 g. Alternatively the range may be 1.4 to 3 g. Alternatively the range may be 1.6 to 3 g. Alternatively the range may be 1.8 to 3 g. Alternatively the range may be 2 to 3 g. Alternatively the range may be 2.2 to 3 g. Alternatively the range may be 2.4 to 3 g. Alternatively the range may be 2.6 to 3 g. Alternatively the range may be 2.8 to 3 g.

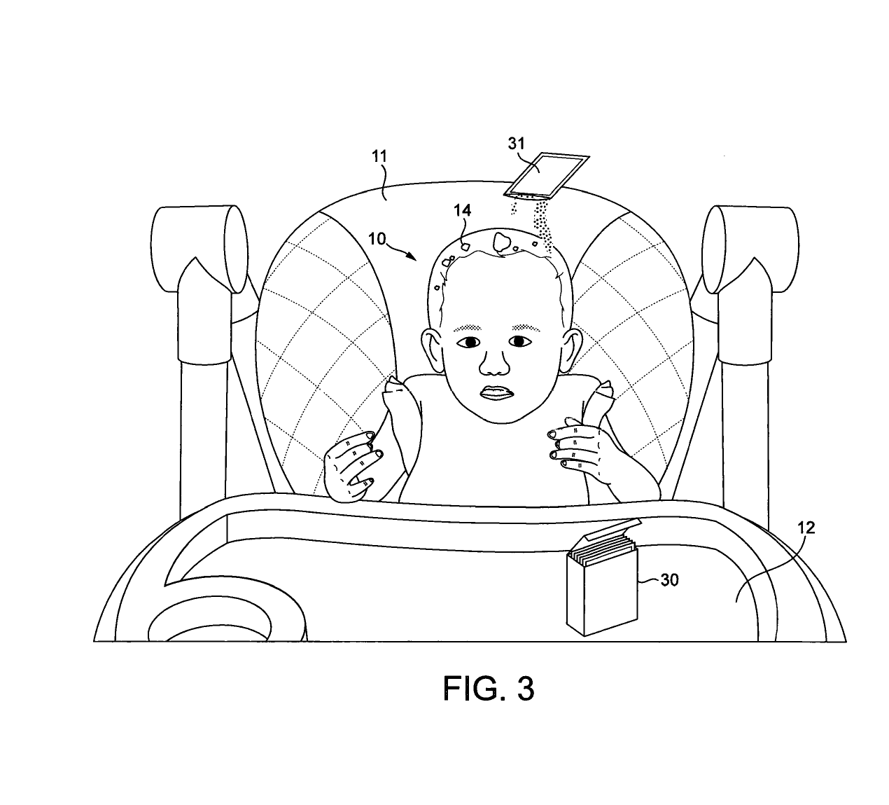

FIG. 3 shows another method of use of the dry powder composition. The composition is stored in a packet 30 of single use sachets 31.
Single use sachets are useful because they can be easily transported in a parent's wallet, pocket, purse, handbag or the like since a sachet is not bulky. Hence, the parent can have a sachet of the composition available when away from home.

The sachet 31 can be opened by tearing away part of the sachet and sprinkling the powder onto the hair of the child. The sachet typically contains around 0.2 to 3 g of the composition which is suitable for a child with short hair. If the child has more hair or longer hair, then more sachets may be used.

Alternatively the weight range of composition in the sachet may be 0.2 to 2.5 g. Alternatively the range may be 0.2 to 2.0 g. Alternatively the range may be 0.2 to 2 g. Alternatively the range may be 0.2 to 1.5 g. Alternatively the range may be 0.2 to 1 g. Alternatively the range may be 0.2 to 0.5 g. Another range may be 0.4 to 3 g. Alternatively the range may be 0.6 to 3 g. Alternatively the range may be 0.8 to 3 g. Alternatively the range may be 1.0 to 3 g. Alternatively the range may be 1.2 to 3 g. Alternatively the range may be 1.4 to 3 g. Alternatively the range may be 1.6 to 3 g. Alternatively the range may be 1.8 to 3 g. Alternatively the range may be 2 to 3 g. Alternatively the range may be 2.2 to 3 g. Alternatively the range may be 2.4 to 3 g. Alternatively the range may be 2.6 to 3 g. Alternatively the range may be 2.8 to 3 g.

Whichever method is used to put the composition onto the hair, the composition is then distributed through the hair to remove any excess grease and foodstuffs which have been deposited on the child's hair. The distribution through the hair can be achieved by any one or more of: massaging; rubbing; ruffling; or working into the hair for 2 to 3 minutes, then a hairbrush or comb can be used to remove the composition and the grease and foodstuffs from the hair.

### Method of Preparation

In order to determine the proportions of the ingredients which provide a composition that is easy to apply uniformly onto the hair, that is effective in cleaning the hair, that is easy to remove from the hair after cleaning, and that achieves a good feel of the hair afterwards, a number of compositions were made using different proportions of ingredients.

The compositions were made in a lidded container. The dry ingredients of starch, baking soda and, where used, conditioning agent were shaken together in the container for around 30 seconds. Then the tests were carried out on a volunteer's hair. The volunteer had left her hair without washing for a few days then smeared yogurt into her greasy hair. The volunteer then parted her hair into sections to test different examples of the composition.

The starches used were corn starch and tapioca flour; baking soda was added as a cleaning agent and the conditioning agent was cucumber extract. In some tests a fragrance was added; it was chamomile in an essential oil form. The technical names of each ingredient are: corn starch—zea mays flour; tapioca flour—Manihot esculenta Crant; baking soda—sodium bicarbonate; cucumber extract—cucumis sativus fruit extract; and chamomile—anthemis nobilis (Roman chamomile) flower oil which naturally contains limonene.

### Results

Tables 1 to 5 are the summary of the results of testing the samples with different levels of the ingredients. The samples were tested for:

a) ease of application of the dry shampoo composition onto the hair;

b) effectiveness in cleaning the hair;

c) ease of removal of the composition after cleaning; and

d) hair feel after use.

The ease of application relates to how easily the composition could be sprinkled onto the hair, i.e. whether or not it was finely textured and could be applied onto the hair uniformly. A combination of corn starch and tapioca flour was found to be easily applied to the hair and equally easy to remove. All the samples tested were found to be equally easy to apply to the hair.

The dry shampoo composition samples were also tested and scored out of 10 for effectiveness in removing grease and food from the hair. The effectiveness in cleaning the hair was found to depend on the percentage of baking soda in the composition.

Once the dry shampoo composition was worked into the hair to clean it, the different compositions were tested for ease of removal of the composition, i.e. if they required a lot of brushing or left a residue even after brushing. All the samples tested were found to be equally easy to remove and left no residue on the hair after use.

After removal of the composition from the hair, the hair feel was assessed and scored out of 10.

The percentages of the components of starch and baking soda, in proportions by weight relative to the total weight of the composition, are set out in Tables 1 to 5 below; scores out of 10 were given for the tests of: effectiveness in cleaning the hair; and the hair feel after use. A score of 1 was given for ineffectiveness in any of the tests and a score of 10 for being highly effective.

TABLE 1
Cucumber Extract: 3%

| | Composition/% weight | Effectiveness in | |
|---|---|---|---|
| Example | relative to the weight | cleaning hair | Hair Feel |
| Number | of the total composition | Score out of 10 | Score out of 10 |
| 1 | Starch: 65 | 5 | 2 |
| | Baking soda: 32 | | |
| 2 | Starch: 70 | 5 | 2 |
| | Baking soda: 27 | | |
| 3 | Starch: 75 | 5 | 3 |
| | Baking soda: 22 | | |
| 4 | Starch: 80 | 5 | 3 |
| | Baking soda: 17 | | |
| 5 | Starch: 85 | 6 | 4 |
| | Baking soda: 12 | | |
| 6 | Starch: 90 | 7 | 6 |
| | Baking soda: 7 | | |
| 7 | Starch: 95 | 6 | 5 |
| | Baking Soda: 2 | | |

TABLE 2
Cucumber Extract: 6%

| | Composition/% weight | Effectiveness in | |
|---|---|---|---|
| Example | relative to the weight | cleaning hair | Hair Feel |
| Number | of the total composition | Score out of 10 | Score out of 10 |
| 1 | Starch: 65 | 4 | 4 |
| | Baking soda: 29 | | |
| 2 | Starch: 70 | 4 | 4 |
| | Baking soda: 24 | | |
| 3 | Starch: 75 | 5 | 4 |
| | Baking soda: 19 | | |
| 4 | Starch: 80 | 6 | 4 |
| | Baking soda: 14 | | |
| 5 | Starch: 85 | 6.5 | 4 |
| | Baking soda: 9 | | |
| 6 | Starch: 90 | 5 | 4 |
| | Baking soda: 4 | | |

TABLE 3
Cucumber Extract: 9%

| | Composition/% weight | Effectiveness in | |
|---|---|---|---|
| Example | relative to the weight | cleaning hair | Hair Feel |
| Number | of the total composition | Score out of 10 | Score out of 10 |
| 1 | Starch: 65 | 6 | 5 |
| | Baking soda: 26 | | |
| 2 | Starch: 70 | 6 | 5 |
| | Baking soda: 21 | | |
| 3 | Starch: 75 | 5 | 5 |
| | Baking soda: 16 | | |
| 4 | Starch: 80 | 7 | 6 |
| | Baking soda: 11 | | |
| 5 | Starch: 85 | 7 | 7 |
| | Baking soda: 6 | | |
| 6 | Starch: 90 | 7 | 8 |
| | Baking soda: 1 | | |

TABLE 4
Cucumber Extract: 12%

| | Composition/% weight | Effectiveness in | |
|---|---|---|---|
| Example | relative to the weight | cleaning hair | Hair Feel |
| Number | of the total composition | Score out of 10 | Score out of 10 |
| 1 | Starch: 65 | 5 | 4 |
| | Baking soda: 23 | | |
| 2 | Starch: 70 | 7 | 5 |
| | Baking soda: 18 | | |
| 3 | Starch: 75 | 7 | 5 |
| | Baking soda: 13 | | |
| 4 | Starch: 80 | 6 | 6 |
| | Baking soda: 8 | | |
| 5 | Starch: 85 | 6 | 6 |
| | Baking soda: 3 | | |

TABLE 5
Cucumber Extract: 15%

| | Composition/% weight | Effectiveness in | |
|---|---|---|---|
| Example | relative to the weight | cleaning hair | Hair Feel |
| Number | of the total composition | Score out of 10 | Score out of 10 |
| 1 | Starch: 65 | 9 | 7 |
| | Baking soda: 20 | | |
| 2 | Starch: 70 | 9 | 9 |
| | Baking soda: 15 | | |
| 3 | Starch: 75 | 10 | 10 |
| | Baking soda: 10 | | |
| 4 | Starch: 80 | 10 | 10 |
| | Baking soda: 5 | | |

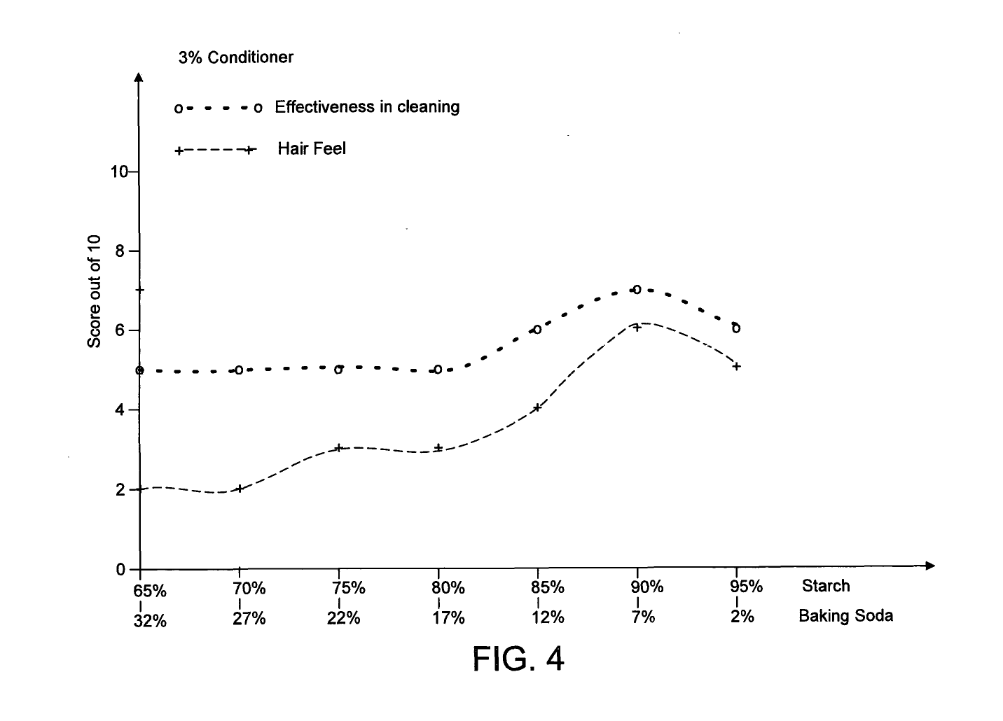

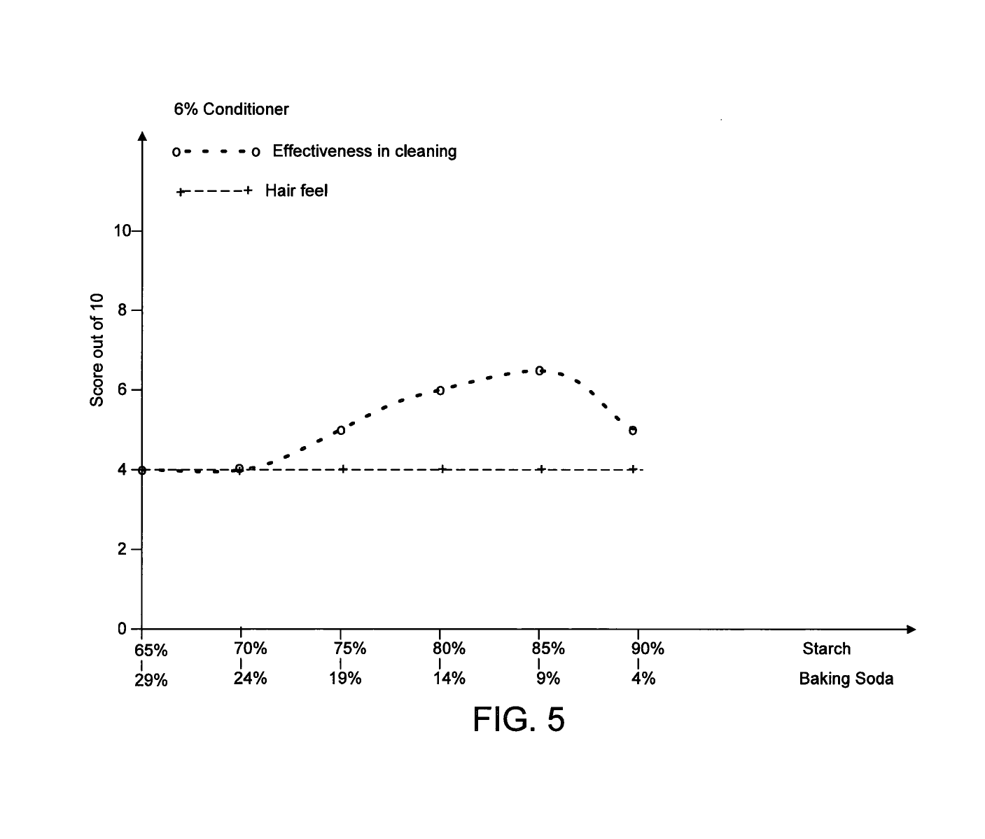

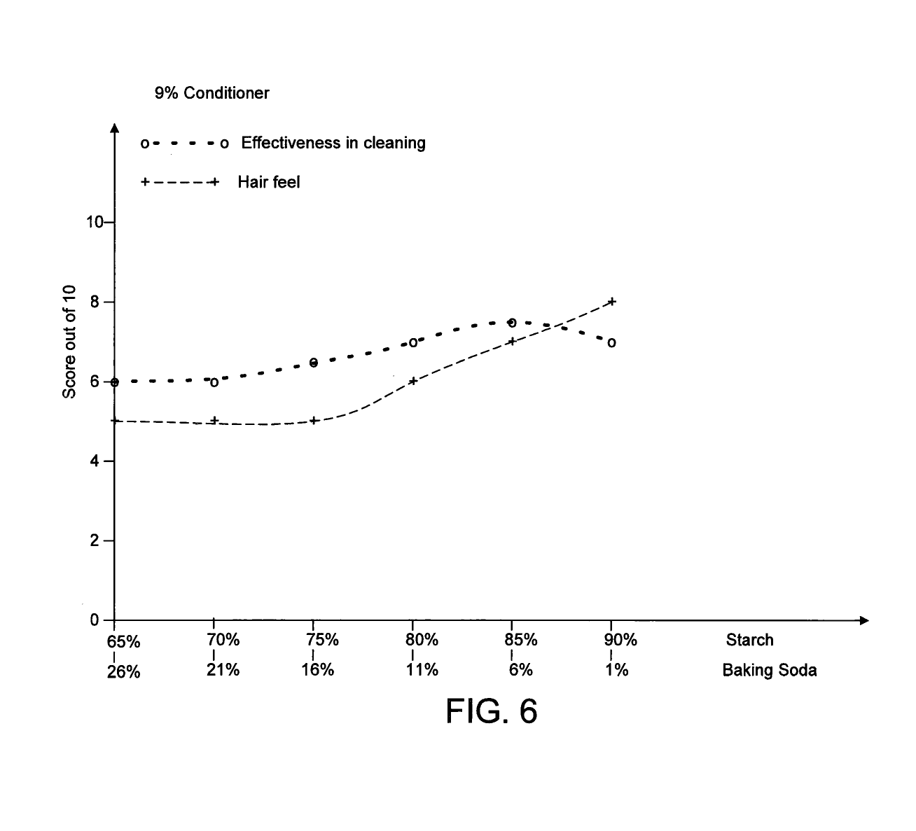

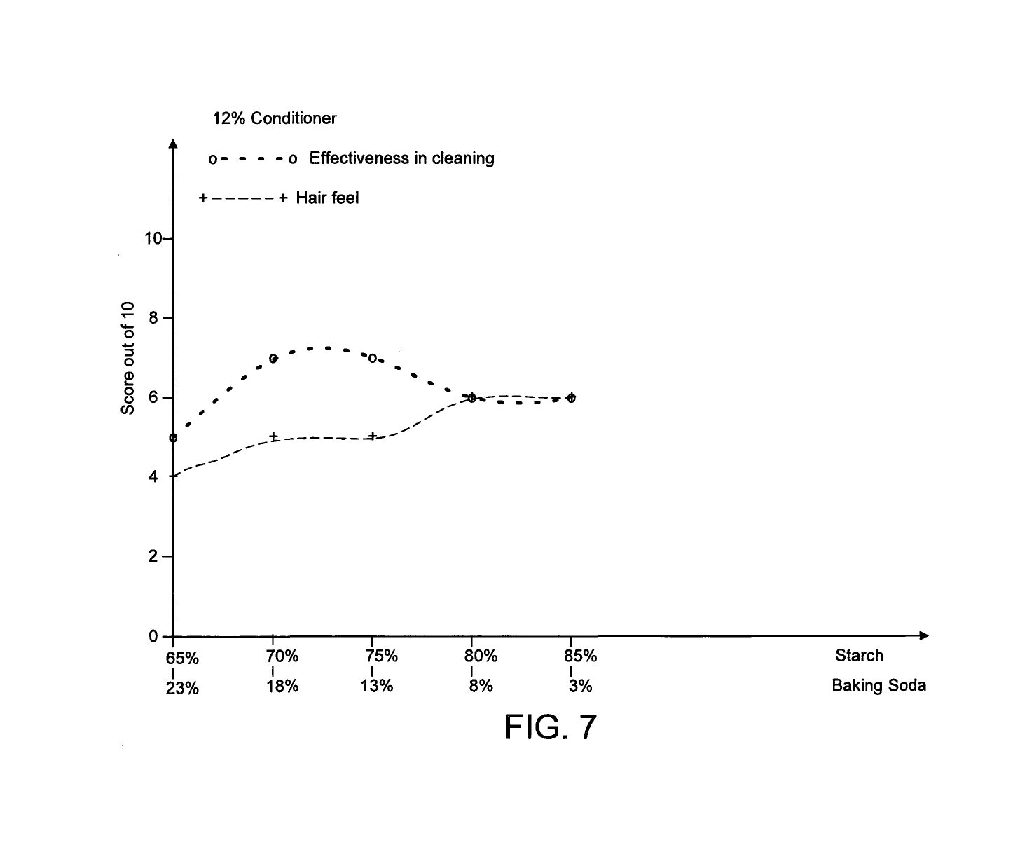

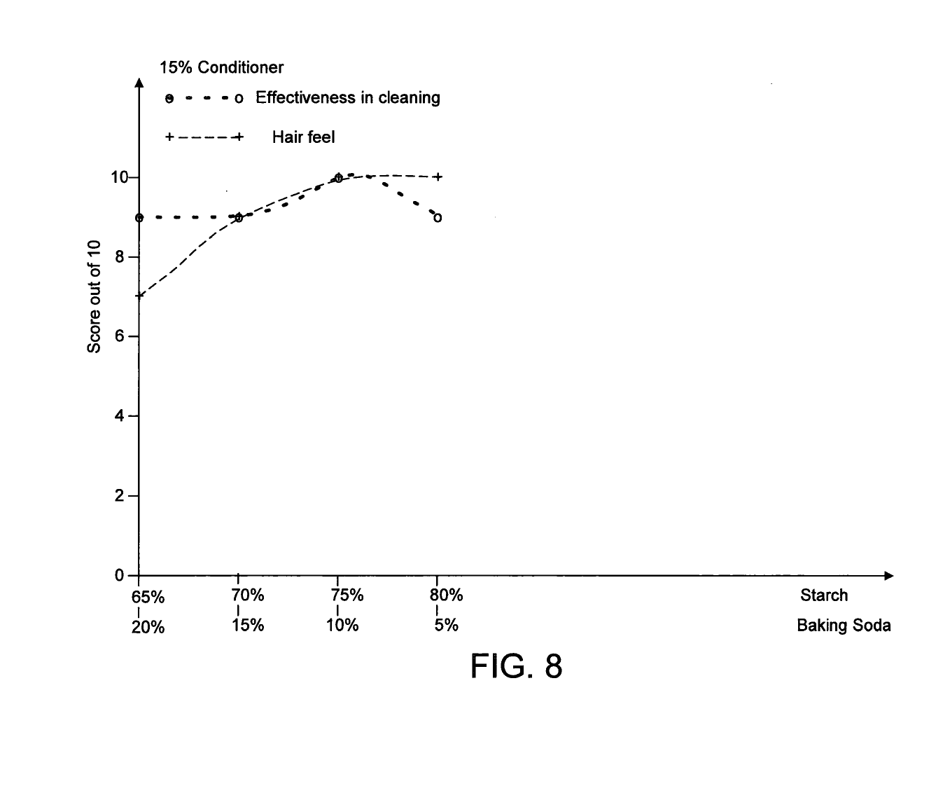

The results listed in the tables were plotted as graphs which are shown in FIGS. 4 to 8. The vertical axis in each of the graphs represents the score out of 10 that was given for each of the tests on the sample compositions.

The horizontal axis represents the percentage of starch in the compositions and also the percentage of baking soda in the compositions.

All percentages of the cucumber extract, starch and baking soda are expressed as proportions by weight relative to the total weight of the composition.

It can be clearly seen that the effective range of starch is 65 to 96%. An effective range for the baking soda is from 1 to 20%, both being in proportions by weight relative to the total weight of the composition.

Although only cucumber extract was tested, other conditioning agents like coconut oil extract or a combination of cucumber and coconut oil extract can be used. The two conditioning agents could be in equal measures but, of course, more or less of one or the other is equally viable.

Although not specifically scored, the level of conditioner that left the hair soft and not overly-limp, was found to be around 3 to 15%. Less conditioner left the hair a little dry and over around 15% left the hair a little too soft and over limp. The level of conditioner did not affect the ease of application, effectiveness in cleaning nor ease of removal of the composition from the hair after use.

Although in the above examples the dry shampoo was stored in a lidded container, the top of the container being provided with perforations, the dry shampoo may be stored in any suitable container. For examples, the top of the container could have a mesh structure like a sieve. Where no holes are present in the top of the container, the composition can be applied to a child's hair by sprinkling the composition on its head using pinches of the composition taken from the container. Or the composition can be put onto the hand of the adult using the shampoo and then applied to the child's hair.

Where the starch is comprised of more corn starch and less tapioca flour, the container may contain a desiccant to keep the composition dry, since corn oil starch has a tendency to absorb moisture from the atmosphere.

Alternatively the composition may be stored in sachets. Single use sachets can be extremely convenient when away from home.

The composition of the present invention, advantageously, contains no harsh or other chemicals which may damage or harm young and/or sensitive skin. It can be used as a dry shampoo composition for hair or skin. The composition provides a rapid solution to the problem of dirty, greasy or otherwise soiled hair and/or skin, particularly suitable, but not limited to, use on children.

The composition can also be used as a dry cleanser for dirty skin when wet washing is not readily available. One method of use is to apply the composition onto the skin, massage onto the skin and remove the composition by the act of wiping with a dry cloth or something similar like a tissue or kitchen paper.

Of course, the present composition is not limited for use on children and those that have sensitive scalps/skin but in fact may be used by all who have little or no time to wet wash their hair or who do not want to wet wash their hair too often.

The composition can equally be used on animals with fur, for example when a pet has become grimy but there is insufficient time to bath the pet and dry it, or when the pet does not enjoy being bathed but tolerates dry grooming.

1-34. (canceled)

35. A dry shampoo composition for cleaning hair, the composition comprising the following constituents in proportions by weight relative to the total weight of the composition:

| | |
|---|---|
| 1 to 20% | baking soda; |
| 3 to 15% | conditioning agent; and |
| 65 to 96% | starch. |

36. The composition according to claim 35, wherein:
the starch component is present in proportions by weight relative to the total weight of the composition in the range 72 to 96%, and the ratio of conditioning agent to baking soda in proportions by weight relative to the total weight of the composition is in the range 1.5:1 to 1:2.5.

37. The composition according to claim 35, the composition additionally comprising a fragrance and wherein the following constituents in proportions by weight relative to the total weight of the composition are:

| | |
|---|---|
| 1 to 20% | baking soda; |
| 3 to 15% | conditioning agent; |
| 0.01 to 5% | fragrance; and |
| 65 to 95.99% | starch. |

38. The composition according to claim 37, wherein:
the starch component is present in proportions by weight relative to the total weight of the composition in the range 67 to 95.99%, the fragrance is present in proportions by weight relative to the total weight of the composition in the range 0.01 to 5%, and the ratio of conditioning agent to baking soda in proportions by weight relative to the total weight of the composition is in the range 1.5:1 to 1:2.5.

39. The composition according to claim 37, wherein the fragrance comprises an essential oil or dry powder extract of one or more of the following: chamomile, lavender, yarrow, dill, coriander, neroli, mandarin, grapefruit, aniseed, beetroot, calendula, neeme, lemon, ginger, rosemary, and cocoa.

40. The composition according to claim 35, wherein at least one of the starch, baking soda and conditioning agent is present in the composition as a dry powder.

41. The composition according to claim 35, wherein the starch comprises one or more of: corn starch, rice starch, tapioca flour, oat flour and potato starch.

42. The composition according to claim 35, wherein the starch comprises a mixture of 95 to 99.9% tapioca flour and 0.1 to 5% corn starch in proportions by weight relative to the total weight of the starch.

43. The composition according to claim 35, wherein the conditioning agent comprises one of or a combination of: cucumber extract and coconut oil extract.

44. The composition according to claim 35, wherein the starch is present in the range 84 to 96%, the baking soda is present in the range 2 to 12%, and the conditioning agent is present in the range from 2 to 4%, all in proportions by weight relative to the total weight of the composition.

45. The composition according to claim 35, wherein the starch is present in the range 77 to 84%, the baking soda is present in the range 10 to 15%, and the conditioning agent is present in the range from 6 to 8%, all in proportions by weight relative to the total weight of the composition.

46. The composition according to claim 35, wherein the starch comprises 0.1 to 5% cornstarch and 95 to 99.9 tapioca flour and the total starch is present in the range 86 to 77%, the baking soda is present in the range 8 to 15%, and the conditioning agent is present in the range from 6 to 8%, all in proportions by weight relative to the total weight of the composition.

47. The composition according to claim 35, wherein the starch is present in the range 83 to 68%, the baking soda is present in the range 5 to 15%, and the conditioning agent is present in the range from 12 to 17%, all in proportions by weight relative to the total weight of the composition.

48. The composition according to claim 35, wherein the starch is present in the range 75 to 80%, the baking soda is present in the range 5 to 10%, and the conditioning agent is present in the range 12 to 15%, all in proportions by weight relative to the total weight of the composition.

49. The composition according to claim 35, wherein the starch is present in the range 75 to 80%, the baking soda is present in the range 5 to 10%, and the conditioning agent is 15%, all in proportions by weight relative to the total weight of the composition.

50. A method of cleaning hair using a composition comprising the following constituents in proportions by weight relative to the total weight of the composition:
1 to 20% baking soda,
3 to 15% conditioning agent, and
65 to 96% starch,
the method comprising:
applying the composition to the hair; and
removing the composition from the hair.

51. The method according to claim 50, wherein the composition is applied to the hair by one or more of:
sprinkling the composition onto the hair;
dispensing pinches of the composition onto the hair; and
dispensing the composition into the palm of a hand and then depositing the composition onto the hair from the hand.

52. The method according to claim 50, further comprising the step of:
distributing the composition through the hair.

53. The method according to claim 50, wherein the composition is further distributed through the hair, after applying to the hair, by one or more of: massaging, ruffling, rubbing, or working into the hair, before removal of the composition.

54. The method according to claim 50, wherein the composition is removed from the hair by brushing or combing.
